(19) United States
(12) Patent Application Publication
(10) Pub. No.: US 2009/0016999 A1
(43) Pub. Date: Jan. 15, 2009

(54) Embryonic cell compositions for wound treatment

(76) Inventors: Michael Cohen, West Orange, NJ (US); Jacob Cohen, West Orange, NJ (US)

Correspondence Address: Pearl Cohen Zedek Latzer, LLP, 1500 Broadway, 12th Floor, New York, NY, 10036, US

(21) Appl. No.: 12/216,971

(22) Filed: Jul. 14, 2008

Related U.S. Application Data
(60) Provisional application No. 60/929,834, filed on Jul. 13, 2007.

(51) Int. Cl. A61K 35/12; A61P 17/02

(52) U.S. Cl. 424/93.7

(57) ABSTRACT

Compositions including formulations comprising stem cells, such as umbilical cord blood stem cells, or embryonic germ cell derivatives, or embryonic stem cells, are provided for enhancement of wound healing. Methods for using the compositions and formulations for enhancing would healing are also provided. Wounds to both soft and bony tissues are encompassed, and include wounds created by surgical procedures.

### CROSS-REFERENCE TO RELATED APPLICATION

This application claims priority under 35 U.S.C. § 119(e) to U.S. provisional patent application Ser. No. 60/929,834, filed Jul. 13, 2007, and is incorporated herein by reference in its entirety.

### BACKGROUND

Wounds are internal or external bodily injuries or lesions caused by physical means, such as mechanical, chemical, viral, bacterial, fungal and other pathogenic organisms, or thermal means, which disrupt the normal continuity of tissue structure. Such bodily injuries include contusions, wounds in which the skin is unbroken, incisions, wounds in which the skin is broken cutting instrument, and lacerations, wounds in which the skin is broken by a dull or, blunt instrument. Wounds can be caused by accident, autoimmune processes, pathological organisms, or created during any surgical procedures.

People afflicted with long-term illness run the risk of getting bed sores, pressure sores and a myriad of skin irritations and chronic wounds. Cancer patients, in particular breast cancer patients, treated with radiation face the risk of skin burns. Wound healing after surgical intervention has been historically problematic. Healing of skin grafts and of plastic surgery procedures are susceptible to poor or slow healing. The benefits of surgery, even in life threatening situations, are offset by the formation of disfiguring scar tissue. Adult wound healing is characterized by fibrosis, scarring, and sometimes by contracture.

Wound healing consists of a series of processes whereby injured tissue is repaired, specialized tissue is regenerated, and new tissue is reorganized. Wound healing consists of three major phases: a) an inflammation stage (0-3 days), b) proliferation stage (3-12 days), and c) a remodeling phase (3 days to 6 months). During the inflammation phase, platelet aggregation and clotting from a matrix which traps the plasma proteins and blood cells to induce the influx of various types of cells. During the cellular proliferation phase, new connective or granulation tissue and blood vessels are formed. During the remodeling phase, granulation tissue is replaced by a network of collagen and elastin fibers leading to the formation of scar tissue.

A problematic wound does not follow the normal time table for the healing process as described above. A problematic wound could fail to follow the normal healing process for any number of reasons, including nutrition, vascular status, metabolic factors, age, immune status, drug therapy, neurologic status and psychologic status, among others. Several local factors also play an important role in wound healing, including the presence of necrotic tissue in the area, infection, foreign body presence, degree of desiccation, presence of edema, pressure, friction, shear maceration and dermatitis.

Methods and compositions for increasing the healing rate and strength of healed wounds, whether external or internal to the body, accidental, pathologic or iatrogenic, and including burns, would be a welcome addition to medical practice.

### SUMMARY

In one embodiment, a composition is provided for the enhancement of wound healing, the composition comprising embryonic germ cell derivatives in a pharmaceutically-acceptable matrix. In another embodiment, the embryonic germ cell derivatives are human embryoid body-derived cells. In another embodiment, the human embryoid body derived cells are LVEC cells or SDEC cells. In other embodiments, the embryonic germ (EG) derivatives are from mouse, pig, chicken, or human. In another embodiment, enhancement of wound healing comprises accelerated rate of epithelial regeneration. In another embodiment, enhancement of wound healing comprises increased wound tensile strength.

In another embodiment, a composition is provided for the enhancement of wound healing, the composition comprising umbilical cord stem cells in a pharmaceutically-acceptable matrix. In a further embodiment, the stem cells are umbilical cord stem cells. In another embodiment, the umbilical cord stem cells are USSC cells. In another embodiment, enhancement of wound healing comprises accelerated rate of epithelial regeneration. In another embodiment, enhancement of wound healing comprises increased wound tensile strength.

In another embodiment, a method for enhancing wound healing is provided comprising applying to a wound site a composition comprising embryonic germ cell derivatives in a pharmaceutically-acceptable matrix. In another embodiment, the embryonic germ cell derivatives are human embryoid body-derived cells. In another embodiment, the human embryoid body derived cells are LVEC cells or SDEC cells. In other embodiments, the embryonic germ (EG) derivatives are from mouse, pig, chicken, or human. In another embodiment, enhancement of wound healing comprises accelerated rate of epithelial regeneration. In another embodiment, enhancement of wound healing comprises increased wound tensile strength.

In another embodiment, a method for enhancing wound healing is provided comprising applying to a wound site a composition comprising stem cells in a pharmaceutically-acceptable matrix. In one embodiment the stem cells are embryonic stem cells. In one embodiment, the stem cells are umbilical cord stem cells. In another embodiment, the umbilical cord stem cells are USSC cells. In another embodiment, enhancement of wound healing comprises accelerated rate of epithelial regeneration. In another embodiment, enhancement of wound healing comprises increased wound tensile strength.

The subject matter regarded as the invention is particularly pointed out and distinctly claimed in the concluding portion of the specification. The invention, however, both as to organization and method of operation, together with objects, features, and advantages thereof, may best be understood by reference to the following detailed description.

### DETAILED DESCRIPTION OF THE EMBODIMENTS

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

This application provides in one embodiment the use of embryonic germ (EG) cell derivatives, and in another embodiment the use of stem cells, that offer benefit in addressing wound healing, and in further embodiments, compositions comprising such cells in a pharmaceutically-acceptable matrix for such uses.

As noted above, wound healing consists of a series of processes whereby injured tissue is repaired, specialized tissue is regenerated, and new tissue is reorganized. Wound healing consists of three major phases: a) an inflammation stage (0-3 days), b) proliferation stage (3-12 days), and c) a remodeling phase (3 days to 6 months). During the inflammation phase, platelet aggregation and clotting from a matrix which traps the plasma proteins and blood cells to induce the influx of various types of cells. During the cellular proliferation phase, new connective or granulation tissue and blood vessels are formed. During the remodeling phase, granulation tissue is replaced by a network of collagen and elastin fibers leading to the formation of scar tissue. The compositions and methods of the invention enhance wound healing typically during at least one of the aforementioned phases, or at two of the three phases, or during all three phases.

Selections of components in the wound healing compositions embodied herein as well as the methods are described in more detail below. These embodiments are merely exemplary and non-limiting.

EG cell derivatives. In the practice of certain of the embodiments herein, human embryonic germ (EG) cell derivatives are used for the wound healing compositions and methods described herein. EG cells can be generated and cultured essentially as described in U.S. Pat. No. 6,090,622. The starting material for isolating cultured embryonic germ (EG) cells is tissues and organs comprising primordial germ cells (PGCs). For example, PGCs may be isolated over a period of about 3 to 13 weeks post-fertilization (e.g., about 9 weeks to about 11 weeks from the last menstrual period) from embryonic yolk sac, mesenteries, gonadal anlagen, or genital ridges from a human embryo or fetus. Alternatively, gonocytes of later testicular stages can also provide PGCs. In one embodiment, the PGCs are cultured on mitotically inactivated fibroblast cells (e.g., STO cells) under conditions effective to derive EGs. The resulting human EG cells resemble murine ES or EG cells in morphology and in biochemical histotype. The resulting human EG cells can be passaged and maintained for at least several months in culture.

Embryoid body-derived cells. In the practice of certain of the embodiments described herein, typically embryoid body-derived cells, derived from embryonic germ cells as mentioned above, are used. Methods for preparing embryoid body-derived cells are described in U.S. Patent Application Publication No. 2003/0175954, published Sep. 18, 2003, and based on Ser. No. 09/767,421, and incorporated herein by reference in its entirety. Such cells can be derived from human embryoid bodies (EBs), which are in turn produced by culturing EG cells, as described above. Methods for making EBs are described below. Unlike EBs, which are large, multicellular three-dimensional structures, embryoid body-derived cells grow as a monolayer and can be continuously passaged. Although EBD cells are not immortal, they display long-term growth and proliferation in culture. Mixed cell EBD cultures and clonally isolated EBD cell lines simultaneously express a wide array of mRNA and protein markers that are normally associated with cells of multiple distinct developmental lineages, including neural (ectodermal), vascular/hematopoietic (mesodermal), muscle (mesodermal) and endoderm lineages. Mesodermal cells include, for example, connective tissue cells (e.g., fibroblasts) bone, cartilage (e.g., chondrocytes), muscle (e.g., myocytes), blood and blood vessels, lymphatic and lymphoid organs cells, neuronal cells, pleura, pericardium, kidney, gonad and peritoneum. Ectodermal cells include, for example, epidermal cells such as those of the nail, hair, glands of the skin, nervous system, the external organs (e.g., eyes and ears) and the mucosal membranes (e.g., mouth, nose, anus, vaginal). Endodermal cells include, e.g., those of the pharynx, respiratory tract, digestive tract, bladder, liver, pancreas and urethra cells. The growth and expression characteristics of EBD cells reveal an uncommitted precursor or progenitor cells phenotype.

Human embryoid bodies (EBs) form spontaneously in human primordial germ cell-derived stem cell cultures that have been maintained in the presence of leukemia inhibitory factor (LIF) (e.g., human recombinant leukemia inhibitory factor) at about, e.g., 1000 units/ml, basic fibroblast growth factor (bFGF), at about 1 ng/ml, and forskolin at about 10 μM for greater than about one month, and, in some situations, as long as three to six months. EBs are also formed when these factors are withdrawn. Additional factors can be added to enhance or direct this process, including, but not limited to, retinoic acid, dimethylsulfoxide (DMSO), cAMP elevators such as forskolin, isobutylmethylxanthine, and dibutryl cAMP, cytokines such as basic fibroblast growth factor, epidermal growth factor, platelet derived growth factor (PDGF and PDGF-AA) nerve growth factor, T3, sonic hedgehog (Shh or N-Terminal fragment), ciliary neurotrophic factor (CNTF), erythropoeitin (EPO) and bone morphogenic factors. The foregoing list is merely exemplary and not intended at be limiting.

Moreover, and as will be discussed further below, embryoid body-derived cells used in the practice of the embodiments herein include cells as described above as well as those that can be transformed or infected. Guidance for methods of so doing may be found in U.S. Patent Application Publication 2003/0175954. Genetic manipulation for the purposes described herein include those that increase the secretion of products beneficial for the treatment of skin and its various aspects as described above.

By way of non-limiting example as to the preparation of EG cell derivatives, EBs are physically removed from the stem cell culture medium where they are formed (see above), and placed in a calcium and magnesium-free phosphate-buffered saline (PBS). The EBs are then sorted into categories by gross morphology, e.g., cystic or solid. After sorting, the EBs are transferred to a mixture of one mg/ml collagenase and dispase enzyme (Boehringer Mannheim), and incubated for 30 minutes to three hours at 37 C.; during this time they are manually agitated or triturated every about 10 to 30 minutes. Other dissociation treatments can be used, e.g., the individual or combined use of several different types of collagenase, dispase I, dispase II, hyaluronidase, papain, proteinase K, neuraminidase and/or trypsin. Each treatment requires optimization of incubation length and effectiveness; cell viability can be monitored visually or by trypan blue exclusion followed by microscopic examination of a small aliquot of the disaggregation reaction. One collagenase/dispase disaggregation protocol calls for incubation for about 30 minutes at 37 C.; this results in between about 10% and 95% of the EB constituent cells disaggregated into single cells. Large clumps of cell may remain intact.

After disaggregation, one to five mls of growth medium are added to the cells. One exemplary medium comprises EGM2-MV medium (Clonetics/Cambrex) with about 10 to 20% fetal calf serum supplemented with antibiotics, e.g., penicillin and streptomycin. The cell suspension is then centrifuged at about 100 to 500 g for about five minutes. The supernatant is then removed and replaced with fresh growth media. The cells are resuspended and plated into a tissue culture vessel that can be coated with cells or typically a biomatrix. In a typical embodiment, collagen type I is used as the substrate.

EBD cells obtained from 4 to 8 EBs can be resuspended in media, e.g., about three ml media (e.g., RPMI), and plated (e.g., into a 3.5 cm diameter plate) onto a surface that has been coated with a collagen (e.g., human type I collagen). The culture media is replaced every two to three days. This is a general method that will allow a wide variety of cell types to proliferate.

In one embodiment EBDs are utilized for the wound healing applications herein. As described above, EBD cells can be clonally isolated and are capable of robust and long-term proliferation in culture. EBD cells are grown and maintained in culture medium or growth medium. Examples of suitable culture media include EGM2-MV medium as mentioned above, knockout DMEM (from GibcoBRL, Life Technologies), Hepatostim (BD Biosciences) and DMEM medium containing knockout serum (Invitrogen) or plasminate, to name only a few examples.

LVEC and SDEC cells. In one embodiment, LVEC cells are used in the aforementioned compositions and methods for treating wounds and enhancing wound healing. In another embodiment, SDEC cells are used in the aforementioned compositions. In yet another embodiment, embryoid body derived cells are used in the aforementioned compositions. In still a further embodiment, embryonic germ cells derivatives are used in the aforementioned compositions.

Umbilical cord stem cells. With regard to the embodiments of the invention wherein secreted products from stem cells are used, in the practice of certain of the embodiments herein, human umbilical cord stem cells are used for the wound healing compositions and methods of use herein. They can be grown in accordance with standard protocols, such as, by way of non-limited example, in USSC media (low glucose DMEM with Glutamax, Invitrogen 10567-014), 10⁻⁷ M dexamethasone (Sigma), 100 U/ml penicillin and 0.1 mg/ml streptomycin. In a further example, medium was changed after 48 hrs then every 2-3 days following. On day 14, proliferating cells were passaged 1:3 into new flasks by using 0.25% trypsin/EDTA and neutralized by trypsin neutralization solution. Every 5 to 7 days cells were similarly passaged. The phenotype of passage 5 umbilical cord stem cells was CD31 (2%), CD34 (0%), CD44 (97%), CD50 (0%), CD71 (47%), CD90, (96%), CD106 (0%). Such cell cultures can provide the composition herein for use in wound healing applications.

USSC Cells. In another example, cells useful for the wound healing compositions and methods embodied herein can be obtained as described in Koglar G, Sensken S, Airey J A, Trapp T, Muschen M, Feldhahn N, et al. 2004. A new human somatic stem cell from placental cord blood with intrinsic pluripotent differentiation potential. J. Exp. Med. 200, 123-135. A somatic stem cell population termed USSC was grown adherently and expanded to 10¹⁵ without losing developmental potential. In vitro, umbilical cord stem cells showed homogeneous differentiation into hematopoietic and neural cell lineage. Immunoassay of umbilical cord stem cells showed CD34, CD45, CD106 negative and CD44 and CD90 positive cell phenotype.

In another embodiment, the cells are CD31, CD34, CD50, CD106 negative, and positive for CD44, CD71, CD90. In another embodiment, the umbilical cord blood derived stem cells are fibroblast-like.

In other embodiment, CD34 positive umbilical cord stem cells are used for the purposes embodied herein.

Embryonic Stem Cells. Other stem cells can be used in the practice of the various embodiments of the invention include embryonic stem cells. Embryonic stem (ES) cells are derived from the inner cell mass of preimplantation embryos. ES cells are pluripotent and are capable of differentiating into cells derived from all three embryonic germ layers. The traditional method used to derive mouse and human embryonic stem (ES) cells involves the use of support cells termed feeder cells or layers. These support cells provide a poorly understood set of signals that promote the conversion from blastocyst inner cell mass (ICM) cells to proliferating ES cells. Most commonly, primary cultures of mouse embryo fibroblasts are used as support cells for both mouse and human ES cultures. The requirement for support cells is not lost following derivation, and ES cell cultures are most commonly maintained on feeder layers until differentiation is desired. WO/9920741 describes the growth of ES cells in a nutrient serum effective to support the growth of primate-derived primordial stem cells and a substrate of feeder cells or an extracellular matrix component derived from feeder cells. The medium further includes non-essential amino acids, an anti-oxidant, and growth factors that are either nucleosides or a pyruvate salt. U.S. Pat. No. 6,642,048 reports growth of ES cells in feeder-free culture, using conditioned medium from such cells. U.S. Pat. No. 6,800,480 describes a cell culture medium for growing primate-derived primordial stem cells comprising a low osmotic pressure, low endotoxin basic medium comprising a nutrient serum and an extracellular matrix derived from the feeder cells. The medium further includes non-essential amino acids, an anti-oxidant (for example, beta-mercaptoethanol), and, optionally, nucleosides and a pyruvate salt.

Cell-containing compositions. The various types of cells mentioned above useful for the embodiments herein are provided in a form by which the cells can be placed in proximity to the wound. Such a composition is referred to herein as a matrix. A pharmaceutically-acceptable matrix is generally used to achieve such a proximity, and can be accomplished by any of a number of means which are known to one of skill in the art. In general, a matrix comprising a polymer or other medium is used to encapsulate the cells, providing an environment beneficial to the maintenance and survival of the cells while at the same time taking advantage of their proximity to the wound to provide benefit. Various non-limiting examples will be described below.

For example, formation of complexes between negatively charged polyanions such as alginate and positively-charged polycations such as poly-L-lysine (PLL) to form alginate-poly-L-lysine-alginate (APA) microcapsules is one approach. This is the most widely used method to microencapsulate cells. In other examples, introducing covalent links within the structure of the alginate layer has increased the stability of alginate beads. Covalent links within a semi-permeable layer made of modified poly(allylamine), which plays a role similar to the one played by poly-L-lysine in alginate poly-L-lysine microcapsules is another approach (Chang, S. J., et al., Biocompatible microcapsules with enhanced mechanical strength. J Biomed Mater Res 59(1): p. 118 126, 2002; Lu, M. Z., et al., A novel cell encapsulation method using photosensitive poly(allylamine alpha-cyanocinnamylideneacetate. J Microencapsul 17(2): p. 245 251, 2000; and Lu, M. Z., et al., Cell encapsulation with alginate and alpha-phenoxycinnamylidene-acetylated poly(allylamine). Biotechnol Bioeng 70(5): p. 479 483, 2000). To enhance the microcapsule's resistance, a photodimerizable reactive group is grafted on the polycationic polymer forming the semi-permeable membrane of microcapsules. This functional reactive group has the particularity to dimerize when exposed to light allowing these cationic polymers to form covalent bonds between one another. U.S. Pat. No. 7,128,931 describes a semi-permeable microcapsule comprising: a bead suited to enclose a material; and a semi-permeable layer covering the bead, said semi-permeable layer being made of a polycation cross-linking derivative covalently linked to the bead. Several decades ago, Lim and Sun, Science 210:908 (1980) described cells encapsulated in a membrane that is permeable to cell substrates and cell secretions, but essentially impermeable to bacteria, lymphocytes, and large immunological proteins. The method of microencapsulation described by Lim and Sun involves forming gelled alginate droplets around isolated islet cells, and then adding coats of poly-L-lysine and additional alginate. The inner gelled core of the microcapsule is then liquefied by chelation. The foregoing descriptions are merely exemplary of any number of methods for maintaining cells in a viable and useful format for application to a site as embodied herein.

In another embodiment a hydrogel carrier or dressing can be used. In another embodiment, a biodegradable polymer or other composition comprising the cells is implanted or provided in a surgical site. In other embodiments, in particular for a superficial wound or one that is at least accessible from the surface of the body, the formulation can be applied as a liquid or gel to the skin or spread on the skin and occluded by a bandage or other device to maintain contact for a period of time. In other embodiments, a semisolid hydrogel formulation comprising cells is placed on the wound, and allowed to remain in place. It may be covered with an occlusive bandage or other device to maintain moisture. After a sufficient period of time, the hydrogel material is removed and discarded.

A hydrogel composition can also be used, and can include a biocompatible polymer component. The biocompatible polymer component can include one or more natural polymers, synthetic polymers, or combinations thereof. For example, the biocompatible polymer can be a polyalkylene oxide such as polyethylene glycol (PEG) or polypropylene glycol, or a derivative of PEG including but not limited to carbonates of polyethylene glycol. The hydrogel can be non-ionic, cationic or anionic. Many other hydrogel-forming polymers are known to the skilled practitioner, including those employing monomeric saccharides, amino acids, and others, to name only an exemplary few. Furthermore, various physicochemical properties are known for hydrogels, such as liquids, pastes, and membranes that can be applied to skin, for example. Various other non-limiting examples are described in US Patent Application 2005/0112151.

It may be advantageous to incorporate additional thickening agents, such as, for instance, Carbopol Ultrez, or alternatively, Carbopol ETD 2001, available from the B.F. Goodrich Co. The selection of additional thickening agents is well within the skill of one in the art.

Hydrogels, further to the description above, may comprise poly(N-vinyl lactam), including homopolymers, copolymers and terpolymers of N-vinyl lactams such as N-vinylpyrrolidone, N-vinylbutyrolactam, N-vinylcaprolactam, and the like, as well as the foregoing prepared with minor amounts, for example, up to about 50 weight percent, of one of a mixture of other vinyl monomers copolymerizable with the N-vinyl lactams. Copolymers or terpolymers of poly (N-vinyl-lactam) may comprise N-vinyl-lactam monomers such as vinylpyrrolidone copolymerized with monomers containing a vinyl functional group such as acrylates, hydroxyalkylacrylates, methacrylates, acrylic acid or methacrylic acid, and acrylamides. Of the poly(N-vinyl lactam)homopolymers, the polyvinylpyrrolidone (PVP) homopolymers are preferred. Of the poly(N-vinyl lactam)copolymers, the vinyl pyrrolidone and acrylamide copolymers are typically employed. Of the poly(N-vinyl lactam)terpolymers, the vinylpyrrolidone, vinylcaprolactam, dimethylaminoethyl methacrylate terpolymers are typically used. A variety of polyvinylpyrrolidones are commercially available.

Hydrogels are stable and maintain their physical integrity after absorbing large quantities of liquid. The gels can be sterilized by radiation sterilization, autoclave or exposed to ethylene oxide. The gels are hydrophilic and capable of absorbing many times of their dry weight in water. Wetting, dispersing agents or surfactants as are known in the art may be added. Glycerin in an amount of 0 to 50 wt. %, preferably from about 5 to 40 wt. % may be added to the gel to increase tack, pliability after drying for the gel. Propylene glycol or polyethylene glycol may also be added. Other additives may be combined with the hydrogels including organic salts, inorganic salts, alcohols, amines, polymer lattices, fillers, surfactants, dyes, etc., among other components described herein.

In one embodiment, the embryonic germ (EG) cell derivatives are embryoid body-derived cells. In another embodiment, the embryoid body-derived cells are LVEC cells or SDEC cells. As will be described in the examples below, EBD cultures are named such that the first two letters refer to the EG culture from which it was derived, the third letter indicates the growth media in which it was derived and is maintained and the fourth letter indicates the matrix on which it is grown.

In other embodiments, the embryonic germ (EG) derivatives are from mouse, pig, chicken, or human.

The following examples are intended to illustrate but not limit the invention. While they are typical of those that might be used, other procedures known to those skilled in the art may alternatively be used.

### EXAMPLES

### Example 1

### Derivation of Embryoid Germ Cell Derivatives

Human pluripotent germ cell cultures were derived from primordial germ cells, isolated and cultured as described above and in Shamblott et al., Proc. Natl. Acad. Sci. USA 95:13726-13731, 1998). Four genetically distinct human EG cell cultures were selected to represent the range of developmental stages at which human EG cultures can be initiated, with karyotypes as noted LV (46, XX), SL (46, XY), LU2 (46, XY) and SD (46, XX). These cultures were derived and cultured from 5, 6, 7, and 11 week post-fertilization primordial germ cells (PGCs), respectively. Embryoid bodies (EBs) were formed in the presence of leukemia inhibitory factor (LIF, 1000 U/ml), basic fibroblast growth factor (bFGF, 2 ng/ml), forskolin (10 μM) and 15% fetal calf serum (FCS, Hyclone). During routine growth, 1 to 5% of the multicellular EG colonies formed large fluid-filled cystic EBs that were loosely attached to a remaining EG colony or to the fibroblast feeder layer. Approximately 10 cystic EBs from each culture were dissociated by digestion 1 mg/ml in Collagenase/Dispase (Roche Molecular Biochemicals) for 30 min. to 1 hour at 37 C. Cells were then spun at 1000 rpm for 5 min.

EB constituent cells were then resuspended and replated in growth media and human extracellular matrix (Collaborative Biomedical, 5 μg/cm2), and tissue culture plastic. Cells were cultured at 37 C, 5% CO₂, 95% humidity and routinely passaged 1:10 to 1:40 by using 0.025% trypsin, 0.01% EDTA (Clonetics) for 5 min. at 37 C. Low serum cultures were treated with trypsin inhibitor (Clonetics) and then spun down and resuspended in growth media. Cell were cryopreserved in the presence of 50% FCS, 10% dimethylsulfoxide (DMSO) in a controlled rate freezing vessel, and stored in liquid nitrogen. Exemplary cell culture designations LVEC and SDEC are the cells derived as mentioned above (LV, SD) grown on human extracellular matrix (EC).

### Example 2

### Derivation of Umbilical Cord Stem-Cells

Frozen human umbilical cord blood mononuclear cells were received from Cambrex, thawed according to manufacturer's recommendation and placed into 3 T75 flasks with 12 ml each of USSC media (low glucose DMEM with Glutamax, Invitrogen 10567-014), 10-7 M dexamethasone (Sigma), 100 U/ml penicillin and 0.1 mg/ml streptomycin. Media was changed after 48 hrs then every 2-3 days following. On day 14, proliferating cells were passaged 1:3 into new flasks by using 0.25% trypsin/EDTA and neutralized by trypsin neutralization solution. Every 5 to 7 days cells were similarly passaged. Aliquots of these umbilical cord stem cells (herein abbreviated “UCSC”) were cryopreserved at several passages.

The phenotype of passage 5 UCSC was CD31 (2%), CD34 (0%), CD44 (97%), CD50 (0%), CD71 (47%), CD90, (96%), CD106 (0%).

Passage 6 UCSC were plated onto a collagen I+Gelatin coated 6 well plate. Each well contained between 0.2 to 1 million cells. Cells were irradiated at 3500 RAD. HuES-2 (Harvard line, Passage 27) cells were plated into the coated wells and the media was changed to HuES media (Cowan, C. A. et. al, 2004). Typical hES colonies were observed at all UCSC densities, so 0.25 million cells per well was chosen for all future work. hES cells were passaged every 3-5 days using 0.05 trypsin/EDTA.

In another experiment, frozen UCB mononuclear cells were purchased from Cambrex (2C-150A, lot: O41113, O50737, HO40926, HO41135, HO41708, HO50567, HO51251, HO51254). Generation and expansion of fibroblast-like cells were following the protocol described by Kögler (Kögler et al. 2004). Briefly, UCB mononuclear cells were cultured in low glucose DMEM+glutaMAX™ (Invitrogen) supplemented with 30% FCS, 10-7M dexamethasone (Sigma), 100 U/ml penicillin and 0.1 mg/ml streptomycin. Cells were initially plated at a density of 5×10⁶ cell/ml in T75 flasks and were placed in a humidified atmosphere at 37° C. and 5% CO₂. Expansion of the cells was performed in the same medium but with 5×10⁻⁸M dexamethasone. Cells were split after reaching confluence by disaggregation with 0.05% trypsin/EDTA and replating at a 1:3 expansion.

Five independent fibroblast-like cell colonies were generated from 8 lots of umbilical cord blood derived mononuclear cells (˜1×10⁸ cells per lot). The generation of fibroblast-like cell cultures was genotype dependent. From the 8 lots of umbilical cord blood derived stem cells, each of Lot HO41708 and HO51251 generated 2 fibroblast-like cell lines and Lot HO41708 generated I line, while other 5 lots generated none. Adherent cells had a spindle/fibroblast morphology similar to USSC cells described by Kögler et al. (Kögler et al. 2004; Kögler et al. 2005) but could be cultured for 6-11 passages in vitro, which is less than that of the reported USSC cells (>20 passages). Fibroblast-like cells had a similar immunophenotype to USSC. Both cell cultures are CD31, CD34, CD50, CD106 negative and positive for CD44, CD71, CD90. These characteristics are different from most umbilical cord blood-derived mesenchymal cell lines which are either CD90 negative (Lee et al. 2004) or CD106 positive (Bieback et al. 2004; Tisato et al. 2007). The fibroblast-like cells embodied herein were similar to HES cell-derived fibroblasts (HES-df) and human foreskin fibroblast cells (HFF), in terms of cell morphology and cell surface markers, as described by Stojkovic (Stojkovic et al. 2005) In that they all expressed cell surface markers CD44 and CD90 but lack endothelial-specific cell marker CD31 and mesenchymal cell specific marker CD106.

### Example 3

### Embryonic Stem Cells

HuES-2 were observed growing on Matrigel in the presence of UCSC conditioned media. This conditioned media was prepared by plating UCSC at 1 million cells per 10 cm plate into 12 mls of HuES media. After 24 hrs media was harvested and sterile filtered. Prior to use on huES-2 cells, 8 ng/ml FGF2 was added to the conditioned media.

### Example 3

### Cell Containing Products for Wound Care Uses

Cells described above are encapsulated in an alginic acid matrix, and applied to a wound. Application to a wound provides an enhanced rate of healing and improved properties of the healed wound.

While certain features have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A composition for enhancing the healing of wounds, the composition comprising embryonic germ (EG) cell derivatives in a pharmaceutically-acceptable matrix.

2. The composition of claim 2 wherein the embryonic germ (EG) cell derivatives are embryoid body-derived cells.

3. The composition of claim 2 wherein said embryoid body-derived cells are LVEC cells or SDEC cells.

4. The composition of claim 1 wherein the embryonic germ (EG) derivatives are from mouse, pig, chicken, or human.

5. The composition of claim 1 wherein enhancement of wound healing is increasing the healing rate or strength of healed wounds.

6. A composition for enhancing the healing of wounds, the composition comprising umbilical cord stem cells in a pharmaceutically-acceptable matrix.

7. The composition of claim 6 wherein the umbilical cord stem cells are CD34^{pos}.

8. The composition of claim 6 wherein the umbilical cord blood stem cells are adherent, CD45^{neg}, HLA class II^{neg} stem cells.

9. The composition of claim 8 wherein said adherent, CD45^{neg}, HLA class II^{neg} stem cells are CD34^{neg}, CD106^{neg}, CD44^{pos} and CD90^{pos}, or are CD31^{neg}, CD34^{neg}, CD50^{neg}, CD106^{neg}, and CD44^{pos}, CD71^{pos}, CD90^{pos}.

10. The composition of claim 6 wherein enhancement of wound healing is increasing the healing rate or strength of healed wounds.

11. A composition for enhancing the healing of wounds, the composition comprising embryonic stem cells in a pharmaceutically-acceptable matrix.

12. The composition of claim 11 wherein enhancement of wound healing is increasing the healing rate or strength of healed wounds.

13. A method for enhancing the healing of wounds comprising applying to the wound a composition comprising embryonic germ (EG) cell derivatives in a pharmaceutically-acceptable matrix.

14. The method of claim 13 wherein the embryonic germ (EG) cell derivatives are embryoid body-derived cells.

15. The method of claim 14 wherein said embryoid body-derived cells are LVEC cells or SDEC cells.

16. The method of claim 13 wherein the embryonic germ (EG) derivatives are from mouse, pig, chicken, or human.

17. The method of claim 13 wherein enhancement of wound healing is increasing the healing rate or strength of healed wounds.

18. A method for enhancing the healing of wounds comprising applying to the wound a composition comprising umbilical cord stem cells in a pharmaceutically-acceptable matrix.

19. The method of claim 18 wherein the cells are CD34^{pos}.

20. The method of claim 18 wherein the umbilical cord blood stem cells are adherent, CD₄₅^{neg}, HLA class II^{neg} stem cells.

21. The method of claim 20 wherein said adherent, CD₄₅^{neg}, HLA class II^{neg} stem cells are CD₃₄^{neg}, CD106^{neg}, CD44^{pos} and CD90^{pos}, or are CD31^{neg}, CD34^{neg}, CD50^{neg}, CD106^{neg}, and CD44^{pos}, CD71^{pos}, CD90^{pos}.

22. The method of claim 18 wherein enhancement of wound healing is increasing the healing rate or strength of healed wounds.

23. A method for enhancing the healing of wounds comprising applying to the wound a composition-comprising embryonic stem cells in a pharmaceutically-acceptable matrix.

24. The method of claim 23 wherein enhancement of wound healing is increasing the healing rate or strength of healed wounds.
